(19) United States
(12) Patent Application Publication
(10) Pub. No.: US 2023/0355128 A1
(43) Pub. Date: Nov. 9, 2023

(54) SYSTEM AND METHOD FOR A MAGNETIC RESONANCE IMAGING TECHNIQUE TO IMAGING TISSUE HETEROGENEITY

(71) Applicant: The General Hospital Corporation, Boston, MA (US)

(72) Inventors: Yi-Qiao Song, Newton, MA (US); Kien-Ninh Ina Ly, Newton, MA (US); Susie Y. Huang, Lexington, MA (US); Bruce R. Rosen, Lexington, MA (US); Elizabeth R. Gerstner, Boston, MA (US)

(21) Appl. No.: 18/312,354

(22) Filed: May 4, 2023

Related U.S. Application Data
(60) Provisional application No. 63/338,394, filed on May 4, 2022.

(51) Int. Cl. G06T 7/00; A61B 5/055

(52) U.S. Cl. CPC A61B 5/055; G06T 7/0012; G06T 2207/10092

(57) ABSTRACT

Diffusion-weighted data are acquired with an MRI system. From the diffusion-weighted data, a comprehensive diffusion tensor distribution (CDTD) is generated. The provides a proportional weighting, at the voxel level, of different diffusion tensors that could describe the water diffusion occurring in the voxel. The CDTD provides insight into tissue microstructure without making assumptions about the structure of a diffusion tensor used to characterize diffusion occurring in tissues of interest. Water pool images, corresponding to different subsets of diffusion tensors in the CDTD, may be generated to assess different components of water diffusion in tissue. Classification images can also be generated from the CDTD to depict different clusters of voxels having similar distributions of diffusion tensors.

### CROSS-REFERENCE TO RELATED APPLICATIONS

This application is based on, claims priority to, and incorporates herein by reference for all purposes, U.S. Provisional Pat. Application No. 63/338,394 filed on May 4, 2022.

### STATEMENT REGARDING FEDERALLY SPONSORED RESEARCH

This invention was made with government support under 5U01EB026996-03, K23NS096056, and K12CA090354 awarded by the National Institutes of Health. The government has certain rights in the invention.

### BACKGROUND

Many diffusion magnetic resonance imaging (MRI) methods, such as diffusion weighted imaging (DWI) and diffusion tensor imaging (DTI), restrict the diffusion model to simplify the acquisition or analysis of the diffusion-weighted imaging data. However, these simplified models fail to accurately describe diffusion and fully characterize the heterogeneity of the tissue. Thus, new methods are needed to quantitatively describe tissue microstructure in a computationally feasible way.

### SUMMARY OF THE DISCLOSURE

The present disclosure addresses the aforementioned drawbacks by providing a method for imaging diffusion using magnetic resonance imaging (MRI). The method includes accessing, with a computer system, diffusion-weighted imaging data acquired from a region-of-interest in a subject using an MRI system. A comprehensive diffusion tensor distribution (CDTD) is generated from the diffusion-weighted imaging data using the computer system. The CDTD indicates a proportion of water molecules in each voxel in the region-of-interest whose diffusion corresponds to each of a plurality of diffusion tensors. The CDTD is output with the computer system.

### BRIEF DESCRIPTION OF THE DRAWINGS

FIG. 1 is a flowchart illustrating an example method for generating a comprehensive diffusion tensor distribution (CDTD) from diffusion-weighted imaging data.

FIG. 2 is a flowchart illustrating an example method for analyzing a CDTD, such as by generating various images or maps (e.g., water pool images, classification images) from the CDTD, which may be advantageous for assessing tissue heterogeneity.

FIGS. 3A-3D show example images of a b = 0 image (FIG. 3A) and three water pools images (FIGS. 3B-3D) generated by masking CDTD data of a glioma patient.

FIGS. 4A and 4B shows examples of a b = 0 image (FIG. 4A) and a classification image (FIG. 4B) generated by classification of CDTD data of a glioma patient.

FIG. 5 is a block diagram of an example magnetic resonance imaging (“MRI”) system that can implement the methods described in the present disclosure.

FIG. 6 is a block diagram of an example diffusion imaging system that can implement the methods of the present disclosure.

FIG. 7 is a block diagram of example components that can implement the system of FIG. 6.

### DETAILED DESCRIPTION

Described here are systems and methods for diffusion imaging with a magnetic resonance imaging (MRI) system. Diffusion-weighted data are acquired using a plurality of different acquisition parameters (e.g., diffusion weighting directions, b-values). From the diffusion-weighted data, a comprehensive diffusion tensor distribution (CDTD) is generated. As will be described below in more detail, this CDTD allows for each voxel in an imaging volume to be represented by a distribution of potential diffusion tensors that potentially describe the diffusion of water molecules in that voxel. That is, the CDTD represents a proportional weighting, at the voxel level, of different diffusion tensors that could describe the water diffusion occurring in the voxel. Voxels with a higher weighting for a particular diffusion tensor contain more water molecules whose diffusion can be described by that particular diffusion tensor. Accordingly, the CDTD provides a unique insight into tissue microstructure by enabling a more robust assessment of water diffusion without making assumptions about the structure of a single diffusion tensor to characterize diffusion occurring in the tissues of interest.

In some examples, the systems and methods are described herein in the context of neuroimaging applications, such as glioma imaging. In other examples, the described systems and methods may be applicable in any anatomy, such as breast, prostate, cardiac, etc. Moreover, the application of the disclosed systems and methods is not limited to cancer imaging and may be applied for diagnosis, characterization, disease monitoring, treatment monitoring, etc., for any pathology of interest, or may even be applied to characterize healthy tissue. As other non-limiting examples, the systems and methods may be used as described for active surveillance of cancers (e.g., prostate cancer) or for characterization of diffusion heterogeneity in tissues, such as in the aging brain.

MRI is widely used in all areas of medicine for diagnostics and staging of diseases, such as to monitor the progression of the diseases and treatment. MRI signals and images are obtained from MRI scanners which include equipment such as magnets to produce a static magnetic field, radiofrequency (RF) electronics to generate pulses of RF magnetic fields, and gradient electronics to produce pulses of constant magnetic fields and gradients. This hardware is operated in concert to produce signals that originate from the hydrogen atoms in the samples (or patients) and the signal is then processed to produce images.

Diffusion MRI techniques can be useful to characterize tissue microstructure. Additionally, within the context of imaging tumors, diffusion MRI techniques can be useful for differentiating between different types of tissues within a tumor region, as compared to conventional anatomical MRI techniques. Brain tumors, particularly gliomas, are spatially heterogeneous and include areas of proliferating tumors cells, neovascularization, and vasogenic edema. Following treatment, there can be foci of residual tumor as well as areas of treatment-related effects (e.g., gliosis, fibrosis, hyalinization). Water molecular diffusion is hindered or restricted by the presence of cells, blood vessels, and other structures inside tissues. For example, water molecules inside axons can diffuse relatively freely along the axon axis; however, the diffusion perpendicular to the axon axis is more restricted. As a result, angular measurement of the diffusion coefficient allows MRI to identify axon directions and white matter tracks. A diseased tissue (e.g., cancer) exhibits different microstructures compared to the normal tissue and thus the water diffusion behavior will be altered.

Molecular diffusion properties are reflective of the molecular composition of a material, as well as the physical and fluidic environment. For example, when a water molecule is in a viscous fluid, its diffusion coefficient, D, will be reduced. The diffusion of water molecules in tissue is also affected by the tissue microstructure and environment. For example, when fluid is inside a tissue, the water diffusion will be restricted due to the presence of solid materials or membranes, including macromolecules, cellular structures, and subcellular structures. In these intracellular spaces, the diffusion coefficient can be significantly reduced from its value in the bulk fluid. As a result, measurement of the diffusion coefficient may be used to characterize tissue microstructures, porous materials, and the like.

In general, the diffusion of water molecules in tissue is not symmetrical, meaning that the restriction in one direction may be significantly greater than in it is in others because of tissue microstructure. Accordingly, this anisotropic diffusion may be described by multiple different diffusion coefficients to account for the directional dependence of diffusion. Mathematically, this directional dependence can be represented by the diffusion tensor, which is a symmetric tensor that may be described by the following 3 × 3 matrix:

$$D = \left[\begin{matrix}{D}_{x x} & {D}_{x y} & {D}_{x z} \\ {D}_{y x} & {D}_{y y} & {D}_{y z} \\ {D}_{z x} & {D}_{z y} & {D}_{z z}\end{matrix}\right] \qquad \text{Eq. (1).}$$

Each of the matrix elements in the diffusion tensor represents a diffusion coefficient defined by the x-, y- and z-directions. Elements along the diagonal represent the diffusion coefficient along the respective direction, and the off-diagonal elements represent the degree of correlation between diffusion in the two directions noted by the subscript of the element.

Diffusion is commonly measured using a spin echo sequence with gradient pulses added during the time between the p90 and p180 pulses and between the p180 pulse and acquisition. The magnetization decay due to diffusion can be described by:

$$M \left(b\right) = {M}_{0} exp \left(- b D\right) \qquad \text{Eq. (2);}$$

where D is the diffusion coefficient and b is the diffusion weighting applied by the pulse sequence, particularly the field gradient pulses used. Several signals with different b-values can be obtained in order to determine D. It is also recognized that the diffusion coefficient can be different along different directions in anisotropic tissues, such as white matter. Diffusion tensor imaging (DTI) was developed in order to obtain the diffusion tensor by applying field gradients along different directions to characterize the magnitude and direction of the diffusion.

In a tissue, many different components are often present within a single voxel of the image. These different components exhibit different values of the diffusion coefficient, so that the total signal is the sum of all components, according to:

$$\frac{M \left(b\right)}{{M}_{0}} = {\sum }_{i = 1}^{n} {w}_{i} exp \left(- b D\right) \qquad \text{Eq. (3).}$$

Here Dᵢ is the diffusion coefficient of the i^{th} component, wᵢ is the i^{th} signal weight or proportion of the i^{th} component, and n is the total number of components. For example, it is advantageous in neuroimaging and interpretation to consider three water diffusion components: one is the signal associated with the axon, which is characterized by anisotropic diffusion behavior; the second is the signal associated with the tissue characterized by isotropically restricted diffusion; and the third signal is from the unrestricted water pool. These three water populations, which are often called pools, are motivated by the gross structure of the brain tissue. This simplified model may not provide sufficient detail for analyzing the tissue microstructure. For example, even within the same population, the details of the tissue microstructure may vary, such as axon diameters, axon orientations, and the degree of the restricted diffusion. As a result, it is desirable to expand the simplified model beyond only three components.

Equation (3) belongs to a class of integral equations that are mathematically ill-conditioned and difficult to solve for noisy experimental data. The inversion stability can be improved by imposing constraints on the obtained distributions. For example, the diffusion tensor eigenvalues may be fixed, and the orientation distribution function (ODF) may be solved for. As another example, the model can be restricted to only consider a few components with pre-defined or heavily constrained diffusivities. Other methods apply artificial restrictions to the potential functional forms of the diffusion properties. Although the constraints facilitate data inversion, they often rely on unverified assumptions, which may give rise to errors.

It is an advantage of the systems and methods described in the present disclosure to provide an approach for obtaining a comprehensive diffusion tensor distribution (CDTD) to eliminate the limitations of conventional diffusion tensor imaging and analysis in order to obtain detailed tissue microstructure information. With this approach, the definition of potential diffusion tensor components can be expanded to include both those with and without axial symmetry, which can provide a comprehensive description of the tissue properties.

As described above, the diffusion tensor D is a symmetric tensor, such that D_{xy} = Dyₓ, D_{xz} = D_{zx}, and D_{yz} = D_{zy}. When D is rotated to the coordinate system of its eigenvectors, the D matrix will be a diagonal matrix with three eigenvalues, λ₁, λ₂, and λ₃, and all other elements will be zero. It is common to order the eigenvalues in an ascending order, i.e., λ₁ ≤ λ₂ ≤ λ₃. In this coordinate frame, if two eigenvalues are equal (e.g., λ₁ = λ₂), then the tensor is considered to be axially symmetric. For example, due to the cylindrical symmetry of axons, it is considered that the diffusion tensor for axonal water will be axially symmetric. Furthermore, since the geometric restriction is along directions transverse to the axon axis, the diffusion tensor for axons is generally considered to be characterized by:

$$λ {}_{1} = {λ}_{2} \leq {λ}_{3} \qquad \text{Eq. (4).}$$

Such symmetry with a strong anisotropy (λ₁ = λ₂ « λ₃) has been observed in many white matter regions where the axon bundles are highly aligned. However, the diffusion anisotropy varies throughout the brain tissues. In gray matter regions, the anisotropy can be quite low due to the broad orientation of axons and other tissues. Even in white matter areas, there can be a significant variation of anisotropy.

Thus, in order to properly characterize the brain tissues, a wide range of candidate diffusion tensors λ₁, λ₂, and λ₃, should be explicitly considered without restricting to certain symmetries or numerical values in order to capture all possible diffusion tensors. For example, the diffusion tensors may be constrained by:

$${d}_{m i n} < {λ}_{1} , {λ}_{2} , {λ}_{3} < {d}_{m a x} \qquad \text{Eq. (5);}$$

where dₘᵢₙ and dₘₐₓ are the minimum and maximum values of the diffusion tensor values allowed in the analysis, respectively. These values, dₘᵢₙ and dₘₐₓ, may be defined based on the clinical or other imaging application. As a non-limiting example, dₘₐₓ may be set to dₘₐₓ = 3 ∗ 10⁻⁵ cm²/s, which is approximately the diffusion coefficient of free water at normal body temperature. Similarly, dₘᵢₙ may be set to

$${d}_{m i n} = 0 \text{or} {d}_{m i n} = \frac{{d}_{m a x}}{1000} ,$$

for example.

A few special cases of the diffusion tensor should be mentioned explicitly. For D with three identical eigenvalues (i.e., λ₁ = λ₂ = λ₃), the diffusion tensor is isotropic. In other words, diffusion along any direction will be identical. This is the case for bulk water, or water in very large pores. For example, diffusion in the ventricles of the brain typically exhibits such isotropic behavior. For D with λ₁ = λ₂ < λ₃, it is often called a prolate spheroid. In particular, when the anisotropy is large, the shape is like a needle. For D with λ₁ < λ₂ = λ₃, it is often called an oblate spheroid and the shape is like a pancake. In the standard analysis of diffusion MRI, the use of prolate spheroids to model the axon is common. However, the oblate spheroid and other shapes, especially those with three different eigenvalues λ₁ ≠ λ₂ ≠ λ₃), have not been used.

Within a given voxel of an image, multiple types of tissue may exist. Thus, the MRI signal can be described as a sum of all the different types of tissues, which are characterized by their diffusion tensors, such as:

$$\frac{S \left(b\right)}{S \left(0\right)} = \int f \left(D\right) exp \left[- b : D\right] d D \qquad \text{Eq. (6);}$$

where b, which is described by a tensor, is the diffusion weighting factor that is applied by the MRI pulse sequence, particularly the application of pulsed field gradient pulses. The gradient pulses may be applied with varying amplitudes along different directions. For example, the directions may be based on a Q-Ball method. The expression b: D indicates a tensor product. The integration may be taken over all the independent tensor elements. For example, one way to perform the integral is to characterize the diffusion tensor D by its eigenvalues (λ₁, λ₂, and λ₃) and orientations or corresponding Euler angles (α, β, and γ). Here, f (D) is the comprehensive diffusion tensor distribution (CDTD) function, which is proportional to the volume of water molecules with a specific diffusion tensor D within a given voxel.

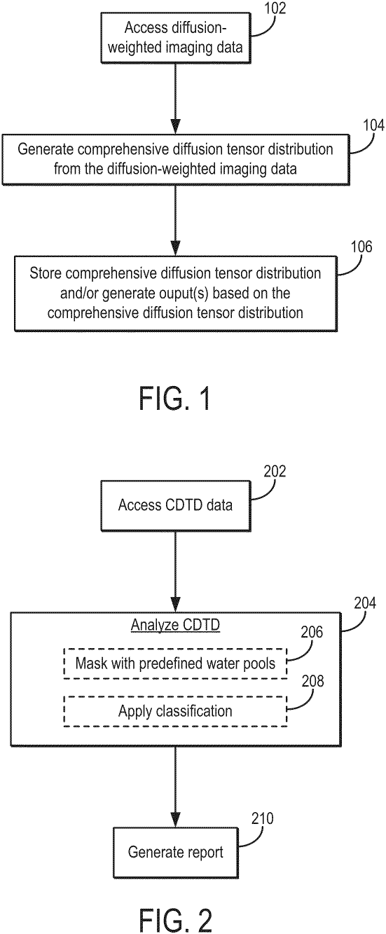

Referring now to FIG. 1, a flowchart is shown as setting forth the steps of an example method for constructing, estimating, or otherwise generating a CDTD. The method includes accessing diffusion-weighted imaging data with a computer system, as indicated at step 102. Accessing the diffusion-weighted imaging data may include retrieving previously acquired data from a memory or other machine-readable data storage device or medium. Additionally or alternatively, accessing the diffusion-weighted imaging data may include acquiring the data with an imaging system and sending, transferring, or otherwise communicating the data to the computer system, which may be a part of the imaging system.

In general, the diffusion-weighted imaging data include diffusion-weighted magnetic resonance imaging data acquired with an MRI system. The diffusion-weighted imaging data may include diffusion-weighted images or k-space, from which diffusion-weighted images may be reconstructed (e.g., as part of step 102). The diffusion-weighted imaging data are acquired while varying a plurality of different acquisition parameters, such as by repeating a diffusion-weighted imaging pulse sequence using combinations of different diffusion-weighting directions and diffusion-weighting factors (i.e., b-values). The diffusion-weighted imaging data may be acquired from a subject, a region-of-interest in a subject, or the like. In some examples, the diffusion-weighted imaging data may be acquired from an object or a material.

In some examples, the diffusion-weighted imaging data may be preprocessed, either before the data are accessed by the computer system or as part of step 102 when accessing the data. For example, as mentioned above when the diffusion-weighted imaging data include raw k-space data, preprocessing may include reconstructing diffusion-weighted images for each b-value and diffusion direction. The preprocessing may also consider and correct various sources of imaging distortion or other artifacts. For example, preprocessing may include gradient non-linearity correction, direction calibration, eddy-current correction, etc. These corrections may be instrument-specific. Data reconstruction and correction are often incorporated routinely into the imaging workflow and may be completed prior to accessing the diffusion-weighted imaging data in step 102, as mentioned above.

In order to obtain the full diffusion tensor information, which includes the magnitude of the diffusion coefficients and the orientation of the eigenvectors, the diffusion-weighted imaging data can be acquired over a wide range of diffusion weighting values (i.e., b-values) and directions of the diffusion-weighting, or diffusion-encoding, gradients used in the pulse sequence(s) implemented by the MRI system to acquire the diffusion-weighted imaging data. As mentioned above, the diffusion-weighted imaging data accessed in step 102 may include data acquired over a wide range of b-values and for a variety of different diffusion directions. For example, in order to obtain high accuracy data for estimating the CDTD, multiple b-value shells may be acquired. As a non-limiting example, eight b-value shells may be used with b-values from 0 to 50,000 s/mm². The large b-value may be useful to identify the components with low diffusion coefficients. In some cases, the maximum b-value may be 10,000 s/mm² or 20,000 s/mm². As a non-limiting example, 32-64 different directions for the gradient orientations may be used. The directions may be uniformly or otherwise distributed around a sphere. Using more directions in the measurement can help improve the angular resolution of the CDTD, however, at the expense of longer scan times.

Since the expected diffusion coefficients span a very large range, for example, from dₘᵢₙ to dₘₐₓ, b-values can be chosen to span a correspondingly large range. For example, the minimum b-value may be 200 s/mm², and the maximum b-value can be 50,000 s/mm². Furthermore, the multiple b-values may be spaced uniformly, logarithmically, or in another fashion.

After accessing the diffusion-weighted imaging data, the CDTD is generated at step 104. For example, the signal for each voxel can be written as a vector, S = {sᵢ}, where i is the index of the experiment covering all b-values and diffusion gradient orientations. Equation (6) may be discretized to a matrix form according to:

$$S = K ⋅ F \qquad \text{Eq. (7);}$$

where S is the vector of diffusion-weighted imaging data, F is the CDTD in a vector form (e.g., Fⱼ is the j^{th} component of the CDTD vector), and K is a kernel function that may be defined as

$${K}_{j} = exp \left(- {b}_{i} : {D}_{j}\right) \qquad \text{Eq. (8).}$$

Here the index i correspond to the b matrix of the i^{th} experiment, and j is the index of the j^{th} diffusion tensor. Each diffusion tensor can be characterized by the three eigenvalues and the three Euler angles:

$${D}_{j} = {D}_{j} \left({λ}_{1} , {λ}_{2} , {λ}_{3} , α , \text{β} , \text{γ}\right) \qquad \text{Eq. (9).}$$

Here, Fⱼ corresponds to the signal weight of the Dⱼ component (i.e., the jth candidate diffusion tensor), and the number of elements of F and D is the same. In other words, Fⱼ represents the proportion of signal within a voxel that can be attributed to the j^{th} component, which has diffusion described by the diffusion tensor Dⱼ.

Step 104 may include discretizing the components of the candidate diffusion tensors, Dⱼ. For example, in order to achieve comprehensive representation of the diffusion tensors, the range of Dⱼ can be quite large. In one non-limiting example, the eigenvalues and Euler angles over which the CDTD is sampled, or otherwise generated, may be discretized as:
λ₁, λ₂, λ₃: each from 0 to 3 ∗ 10⁻³ mm²/s, in 10 steps, and
α, β, γ: each from 0° to 180° or 360°, in 10 steps.

Discretizing λ₁, λ₂, and λ₃ can be done uniformly, logarithmically, or otherwise. In one non-limiting example, the total number of diffusion tensor components can be very large (e.g., 10⁶). The number of components can be reduced significantly by considering that λ₁ ≤ λ₂ ≤ λ₃, which does not limit the shape or symmetry of the distinctive diffusion tensors.

Step 104 can also include solving Equation (7) for each voxel to determine the proportion of water molecules in the voxel whose diffusion corresponds to each of the discretized diffusion tensors. In other words, solving Equation (7) can provide a signal weight for each of the combinations of discretized eigenvectors and Euler angles. For example, the solution to Equation (7) can be obtained by inversion techniques, such as CONTIN or Fast Laplace Inversion (FLI), etc. In some cases, the FLI algorithm can accelerate the inversion computation.

The CDTD generated from the diffusion-weighted imaging data is then stored for later use, may be output with the computer system, or may have other outputs generated based on the CDTD, as indicated at step 106. For example, the CDTD may be stored as CDTD data in a memory of the computer system, or in another machine-readable data storage device or medium. Additionally or alternatively, the CDTD may be output to a user, or one or more outputs may be otherwise generated based on the CDTD. As one example, one or more images or maps representing diffusion associated with different candidate diffusion tensors may be generated and output to a user, as described below in more detail.

Referring now to FIG. 2, a flowchart illustrates the steps of an example method to analyze the CDTD to generate one or more types of output that can represent different aspects of water molecule diffusion occurring in the subject or object for which the CDTD was generated.

The method includes accessed CDTD data with a computer system in step 202. Accessing the CDTD data may include retrieving previously generated data from a memory or other machine-readable data storage device or medium. Additionally or alternatively, accessing the CDTD data may include generating the CDTD data from diffusion-weighted imaging data, for example as described above with respect to FIG. 1, and sending, transferring, or otherwise communicating the data to the computer system.

As described above, the comprehensive diffusion tensor distribution stored in the CDTD data provides a comprehensive characterization of the diffusion properties of tissue, or other material, for each voxel. However, the resulting CDTD data is very large (e.g., half a million data points) and can be difficult to visualize without taking additional steps. Such analysis can be performed in process block 204, which can facilitate the visualization of CDTD data.

In one approach, the analysis in process block 204 can include masking the CDTD in step 206 to generate images that depict selected groups of diffusion tensors in the CDTD. The groups can be defined based on user selected criteria, predefined criteria, or the like. In general, the groups of diffusion tensors selected by the masking can be based on different types of diffusion of interest. As another approach, the analysis in process block 204 can include applying a classification to the CDTD in step 208, which can be used to form a classification image or map. Additional or alternative analyses based on the CDTD data may also be implemented in process block 204. Non-limiting examples of the masking and classification approaches mentioned above are described in more detail below.

Regardless of the analysis method used, a report can be generated in step 210, which can be based on the analysis, or analyses, performed in process block 204. For example, the report can include water pool images (e.g., images formed using the masking process in step 206), classification images or maps (e.g., images formed using the classification process in step 208), other images or maps generated from the CDTD data, or combinations thereof. The report may also include other information, such as information about the number of voxels belonging in each class or water pool, which can be compared over time to assess changes in the subject and/or object. For example, changes in the number of voxels belonging to a particular class and/or water pool may be analyzed to assess the course of treatment being delivered to a subject, or to assess other anatomical or physical changes (e.g., changes caused by degenerative diseases_. The report can also include the full CDTD data, or other subsets of the data.

As described above, one analysis that may be performed in process block 204 is the generation of one or more water pool images, or maps, from the CDTD, as indicated at step 206. The CDTD is a comprehensive description of the diffusion properties of the tissue. To help interpret or visualize the CDTD, various water pools or water groups may be selected for visualization by defining groups of diffusion tensors to view or analyze. The groups may be defined based on threshold values or various symmetries, asymmetries, or other relationships between the eigenvalues. Advantageously, in the disclosed methods, these relationships can be defined or otherwise selected in step 206 for visualization purposes, rather than when constructing the CDTD itself. This allows the CDTD to be computed without requiring limiting assumptions of the forms of the diffusion tensors, a priori, as is done in conventional diffusion imaging methods. Thus, the CDTD is able provide more accurate representation of the water diffusion and tissue heterogeneity.

Different parts of the CDTD correspond to different types of diffusion tensors and their symmetries. Any number of groups or pools of water can be predefined or otherwise selected based on the types of diffusion that are of interest for a given application. For example, the area of large and approximate isotropic diffusion coefficient λ₁ ≈ λ₂ ≈ λ₃) can be chosen and related to the free water. Another area of highly anisotropic diffusion coefficient λ₁ ≈ λ₂ « λ₃) can be chosen and related to the water in axons. Thus, in step 206, one or more groups of water pools can be defined or otherwise selected. The water pools can be described based on minimum or maximum thresholds, such as Dₑᵤₜ,_{1,} D_{cut},₂, and D_{cut},₃, of any of the eigenvalues. They may also be described based on the relationships between the eigenvalues (e.g., λ₁ = λ₂ = λ₃ or λ₁ ≈ λ₂ ≈ λ₃). As a non-limiting example, the groups of water or water pools can be selected according to the groups of parameters outlined in Table 1 below.

| | |
|---|---|
| Water Pool | Associated Diffusion Tensor Parameters |
| First water pool | λ₁, λ₂, λ₃ ≥ D_{cut},₁ and 3λ₁ ≥ λ₂, 3λ₂ ≥ λ₃, |
| | where D_{cut,1} = 0.5 ∗ 10⁻³ mm²/s |
| Second water pool | λ₁ < D_{cut,2}, λ₂ < D_{cut,2}, and λ₁ + λ₂) < $\left({λ}_{1} + {λ}_{2}\right) < \frac{{λ}_{3}}{8} ,$ |
| | where D_{cut,2} = 0.1 ∗ 10⁻³ mm²/s |
| Third water pool | ${λ}_{1} < {D}_{c u t , 3} , {λ}_{2} > \frac{{λ}_{3}}{3} , a n d {λ}_{2} < 3 {λ}_{3} ,$ |
| | where D_{cut,3} = 0.1 ∗ 10⁻³ mm²/s |

The cutoff thresholds may be chosen as any values according to the application. Additionally or alternatively, the water groups may be defined by other relationships between the eigenvalues, by the Euler angles of the diffusion tensors, or combinations thereof. More or fewer definitions of water pools may be used as well.

The first water pool signal in Table 1 corresponds to highly diffusive water molecules, which can be considered less restricted and therefore related to free water. The second water pool signal in Table 1 describes highly anisotropic diffusion tensors (i.e., λ₁ ≈ λ₂ « A₃), which can be related to the water in axons or other highly directional structures. The third water pool signal in Table 1, on the other hand, shows a very different symmetry (i.e., λ₁ < λ₂ ≈ λ₃) and is related to the water in other tissue environments. In practice, any number of other water pools can be defined based on selecting thresholds and/or ranges of parameters to define the diffusion tensors in the CDTD to represent the water pool of interest.

Using the diffusion tensor parameters for a selected water pool, a mask associated with the selected water pool can be generated. Signal from area water pool (p) selected from the CDTD can be obtained by:

$${W}_{p} = \frac{{\sum }_{j} {M}_{j} {F}_{j}}{{\sum }_{j} {F}_{j}} \qquad \text{Eq. [13](10).}$$

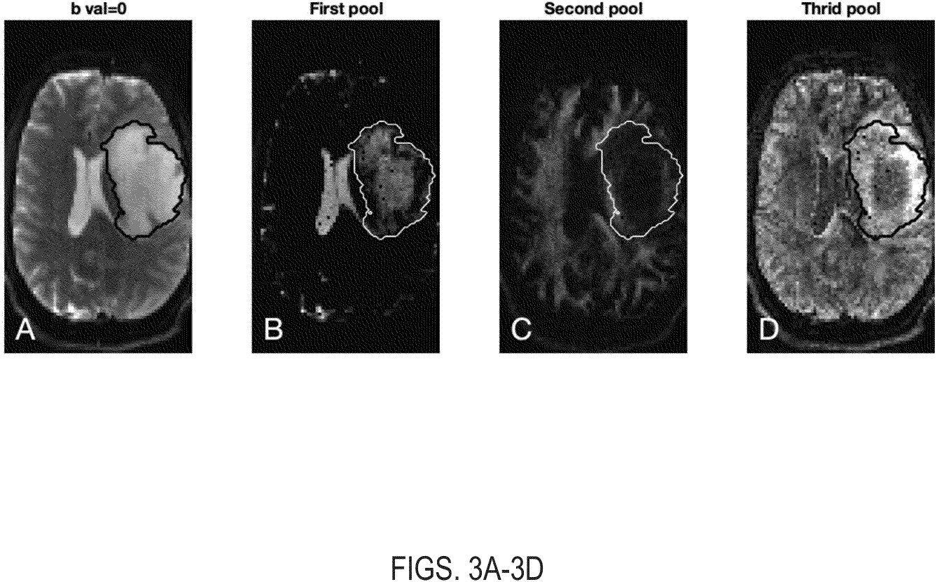

Here, Mⱼ is the mask vector of the same shape as Fⱼ. The mask vector can be determined based on the defined water pools, for example based on the associated diffusion tensor parameters selected to define the water pool of interest. For each candidate diffusion component j, Mⱼ can be set to 1 for diffusion components that fall within a given water pool based on the definitions chosen. This can be repeated for all of the water pools defined. Thus, Equation (10) can be calculated for each voxel to provide the proportion of signal within the voxel that corresponds to each of the pre-defined water pools. A map can be generated for each water pool, as shown, for example, in FIGS. 3A-3D. These maps show the proportion of the signal within each voxel that can be attributed to each water pool; that is, the water diffusion described by the diffusion tensors selected based on the group of diffusion tensor parameters for the respective water pool.

FIGS. 3A-3D show examples of CDTD visualization in a patient with a glioma, which may be generated in step 206 using the masking process described above. A b = 0 s/mm² image (FIG. 3A) is shown alongside images of the three pools of water signals (FIGS. 3B, 3C, and 3D) determined by the CDTD analysis. The definitions of the three water pools represented in FIGS. 3B-3D are consistent with those defined above in Table 1, though other water pool definitions may be used by selecting different groups of parameters, as described above. The black/white lines in FIGS. 3A-3D indicate the perimeter of a cancerous region. The voxel intensity in each of FIGS. 3B-3D represents the proportion of the voxel attributed to the defined water pool. For example, in FIG. 3B, the free water was calculated by Equation (10) using a mask that selects the free water area of CDTD based on the first water pool from Table 1. Likewise, FIGS. 3C and 3D show the same analysis for the second and third water pools from Table 1, respectively.

From such CDTD analysis and by displaying the images and analysis as shown in FIGS. 3A-3D, many interesting features can be observed. For example, the tumor region (delineated by a black line in FIG. 3A) exhibits high signal intensity in the b = 0 s/mm² image. In FIG. 3B, the center of the tumor region shows a high signal from the first water pool, which can be associated free water. In FIG. 3C, the tumor region exhibits a strong reduction of the second water signal compared to the rest of the brain, which indicates that the axonal tissues are being excluded in the center of the tumor. FIG. 3D shows an enhancement of the third water signal along the perimeter of the tumor region.

In this example, it is clear that the center of the tumor region is significantly different from the perimeter or peritumoral area, highlighting the tissue heterogeneity of the tumor region. Conventional clinical MRIs are typically not able to reflect this degree of tumor heterogeneity. It is an advantage of the systems and methods described in the present disclosure that tissue heterogeneity, including tumor heterogeneity, can be better visualized using the CDTD. Because the CDTD provides information related to a plurality of different candidate diffusion tensors, different water pools can be selected and visualized based on the CDTD. That is, assumptions do not need to be made when generating the CDTD, such that any number of different arbitrary water pools can be defined and visualized from the generated CDTD, allowing for a more robust assessment of tissue heterogeneity than available with convention diffusion imaging techniques.

Besides visualizing the various water pools. The pool definitions may be used for other analysis. For example, tumor size may be computed by thresholding the water pool images. The tumor size may be broken down to indicate the proportion of the tumor is attributed to each of the water pools. The water pool images may also be used to calculate metrics of tumor heterogeneity. Additionally or alternatively, a sum of different water pools may be calculated (or an image that is the sum of different water pool images may be generated), a ratio of different water pools may be calculated (or an image that is the ratio of different water pool images may be generated), a difference between different water pools may be calculated (or an image that is the difference between different water pool images may be generated), or the like. Such statistics and others can be compared throughout treatment or for other analysis.

As described above, another analysis that may be performed in process block 204 is the generation of one or more classification images, or maps, from the CDTD, as indicated at step 208. In these instances, statistical methods can be applied to classify the CDTD data into any number of classes (or types) of tissues, materials, or the like. As one example, a clustering analysis can be used to group voxels that are more similar within a cluster than to those in other clusters. A cluster or a combination of clusters may be considered to correspond to a tissue type. Many different algorithms can be used to achieve such a classification, including hierarchical clustering, the k-means algorithm, bi-clustering (also known as co-clustering or two-mode-clustering), neural network or other machine learning methods, and others. Principal component analysis (PCA) may also be used.

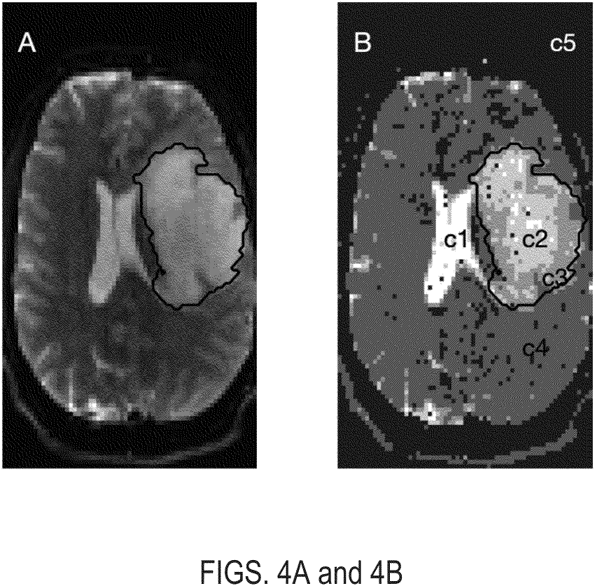

In applying such classification analysis, a cluster identity can be given to each voxel of an image, which can be used to form a classification image. An example of such a classification analysis is shown in FIGS. 4A and 4B, in which a k-means method was applied to the CDTD of the glioma patient as also shown in FIGS. 3A-3D. The classification image, which may be generated in step 208, is illustrated in FIG. 4B, whereas the b = 0 s/mm² image of the subject is again shown in FIG. 4A. In this case, the clustering analysis resulted in five clusters (c1, c2, c3, c4, and c5). The first cluster, c1, highlights the ventricle area where the signal is primarily from free water (e.g., cerebrospinal fluid). The areas of clusters c2 and c3 cover the tumor area, where the second cluster, c2, highlights the central tumor region. The area of the fourth cluster, c4, is the non-tumoral area with healthy brain tissue, and the fifth cluster, c5, is outside of the brain. Compared to the b = 0 image (FIG. 4A), the classification image (FIG. 4B) clearly shows the heterogeneity within the tumor region based on the different clusters of voxels that are generated within the tumor region. For example, the c2 area is distinct from the peritumoral region (c3).

The various clusters can be compared in further analyses to determine or otherwise assess tumor heterogeneity, or to monitor changes in the tumor through surveillance or treatment. Such cluster images can also be used for surgery or treatment planning.

Gliomas originate from glial cells within the central nervous system. Gliomas correspond to about 30% of all brain tumors and central nervous system tumors, and 80% of all malignant brain tumors. Gliomas are spatially heterogeneous (including areas of necrosis, hypoxia, and vasogenic edema) and diffusely infiltrate surrounding normal brain tissue. Thus, even a complete surgical resection will often leave behind microscopic disease. Gliomas can be benign (World Health Organization (WHO) grade I and II) and malignant (WHO grade III and IV). While low-grade gliomas are typically slow growing, high-grade gliomas (including glioblastomas) are highly aggressive and are associated with a dismal prognosis. Accurate detection of tumor extent is important to assist with surgical and radiation therapy planning. Longitudinal monitoring of the tumor resection cavity and residual tumor are important to improve patient outcomes.

Other tumor types may also exhibit microstructural changes during disease progression. For example, prostate cancer is characterized by the progressive degradation of prostate glandular structure. Diffusion MRI is a useful diagnostic method to identify prostate cancer. Because it is a slowly developing cancer, active surveillance can be an effective method for low grade prostate cancer. For such patients, the CDTD may offer an effective method to accurately monitor tumor progression.

There are several ways that CDTD methods, which may be combined with clustering analysis, may be used. Non-limiting examples will be provided below.

For example, CDTD may be used for the first MRI of the patient that is often obtained before surgery or other treatment. CDTD and clustering analysis (or other analyses of the CDTD, including water pool masking) can be performed to assess baseline tumor size and tumor heterogeneity. Subsequent MRI/CDTD/Clustering analysis can be obtained, for example, after certain time intervals from the initial examination, or interventions (e.g., surgery, radiation treatment, oncological treatments, etc.). The baseline and subsequent data for a particular patient can be used to monitor for disease progression, determine the effectiveness of the intervention, and to guide further treatment decisions. Disease progression may appear as enlargement of the tumor regions (e.g., c2 region, c3 regions, or both), changes to the spatial heterogeneity of the tumor (for example, the c3 regions may appear similar to c2 regions such that previously labeled as c3 are subsequently labeled as c2), or development of new tumors regions (e.g., other cluster regions or regions similar to c2 or c3) in brain regions remote from the original tumor. Similarly, the proposed methods may be used to distinguish tumor recurrence from treatment-related changes based on distinct tissue microstructural differences between these two entities.

For a population of patients, the data (MRI/CDTD/clustering) can be combined to aid in the diagnosis of tumor grade (based on the WHO classification) or tumor molecular characteristics. For example, the presence of c2 clusters may be correlated with WHO grade III gliomas while the presence of c3 clusters alone may correlate with WHO grade II tumors.

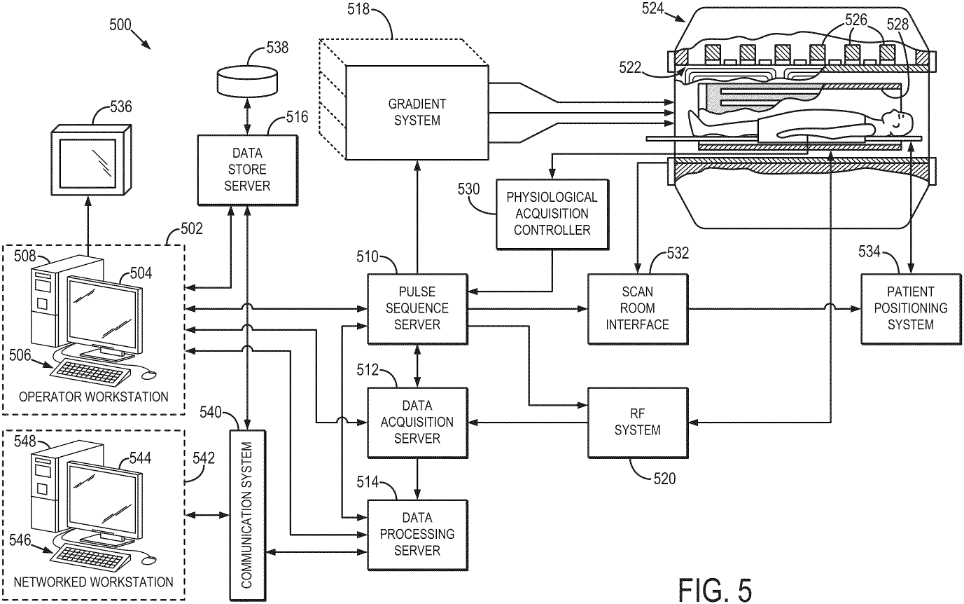

Referring particularly now to FIG. 5, an example of an MRI system 500 that can implement the methods described herein is illustrated. The MRI system 500 includes an operator workstation 502 that may include a display 504, one or more input devices 506 (e.g., a keyboard, a mouse), and a processor 508. The processor 508 may include a commercially available programmable machine running a commercially available operating system. The operator workstation 502 provides an operator interface that facilitates entering scan parameters into the MRI system 500. The operator workstation 502 may be coupled to different servers, including, for example, a pulse sequence server 510, a data acquisition server 512, a data processing server 514, and a data store server 516. The operator workstation 502 and the servers 510, 512, 514, and 516 may be connected via a communication system 540, which may include wired or wireless network connections.

The MRI system 500 also includes a magnet assembly 524 that includes a polarizing magnet 526, which may be a low-field magnet. The MRI system 500 may optionally include a whole-body RF coil 528 and a gradient system 518 that controls a gradient coil assembly 522.

The pulse sequence server 510 functions in response to instructions provided by the operator workstation 502 to operate a gradient system 518 and a radiofrequency (“RF”) system 520. Gradient waveforms for performing a prescribed scan are produced and applied to the gradient system 518, which then excited gradient coils in an assembly 522 to produce the magnetic field gradients (e.g., Gₓ, G_{y}, and G_{z}) that can be used for spatially encoding magnetic resonance signals. The gradient system 518 can also be used to produce diffusion gradients to provide the desired diffusion weighting along any desired direction. The gradient coil assembly 522 forms part of a magnet assembly 524 that includes a polarizing magnet 526 and a whole-body RF coil 528.

RF waveforms are applied by the RF system 520 to the RF coil 528, or a separate local coil to perform the prescribed magnetic resonance pulse sequence. Responsive magnetic resonance signals detected by the RF coil 528, or a separate local coil, are received by the RF system 520. The responsive magnetic resonance signals may be amplified, demodulated, filtered, and digitized under direction of commands produced by the pulse sequence server 510. The RF system 520 includes an RF transmitter for producing a wide variety of RF pulses used in MRI pulse sequences. The RF transmitter is responsive to the prescribed scan and direction from the pulse sequence server 510 to produce RF pulses of the desired frequency, phase, and pulse amplitude waveform. The generated RF pulses may be applied to the whole-body RF coil 528 or to one or more local coils or coil arrays.

The RF system 520 also includes one or more RF receiver channels. An RF receiver channel includes an RF preamplifier that amplifies the magnetic resonance signal received by the coil 528 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{\left({I}^{2} + {Q}^{2}\right)}$$

and the phase of the received magnetic resonance signal may also be determined according to the following relationship:

$$ϕ = {tan}^{- 1} \left(\frac{Q}{I}\right)$$

The pulse sequence server 510 may receive patient data from a physiological acquisition controller 530. By way of example, the physiological acquisition controller 530 may receive signals from a number of different sensors connected to the patient, including electrocardiograph (“ECG”) signals from electrodes, or respiratory signals from a respiratory bellows or other respiratory monitoring devices. These signals may be used by the pulse sequence server 510 to synchronize, or “gate,” the performance of the scan with the subject’s heartbeat or respiration.

The pulse sequence server 510 may also connect to a scan room interface circuit 532 that receives signals from various sensors associated with the condition of the patient and the magnet system. Through the scan room interface circuit 532, a patient positioning system 534 can receive commands to move the patient to desired positions during the scan.

The digitized magnetic resonance signal samples produced by the RF system 520 are received by the data acquisition server 512. The data acquisition server 512 operates in response to instructions downloaded from the operator workstation 502 to receive the real-time magnetic resonance data and provide buffer storage, so that data are not lost by data overrun. In some scans, the data acquisition server 512 passes the acquired magnetic resonance data to the data processor server 514. In scans that require information derived from acquired magnetic resonance data to control the further performance of the scan, the data acquisition server 512 may be programmed to produce such information and convey it to the pulse sequence server 510. For example, during pre-scans, magnetic resonance data may be acquired and used to calibrate the pulse sequence performed by the pulse sequence server 510. As another example, navigator signals may be acquired and used to adjust the operating parameters of the RF system 520 or the gradient system 518, or to control the view order in which k-space is sampled. In still another example, the data acquisition server 512 may also process magnetic resonance signals used to detect the arrival of a contrast agent in a magnetic resonance angiography (“MRA”) scan. For example, the data acquisition server 512 may acquire magnetic resonance data and processes it in real-time to produce information that is used to control the scan.

The data processing server 514 receives magnetic resonance data from the data acquisition server 512 and processes the magnetic resonance data in accordance with instructions provided by the operator workstation 502. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 514 are conveyed back to the operator workstation 502 for storage. Real-time images may be stored in a data base memory cache, from which they may be output to operator display 502 or a display 536. Batch mode images or selected real time images may be stored in a host database on disc storage 538. When such images have been reconstructed and transferred to storage, the data processing server 514 may notify the data store server 516 on the operator workstation 502. The operator workstation 502 may be used by an operator to archive the images, produce films, or send the images via a network to other facilities.

The MRI system 500 may also include one or more networked workstations 542. For example, a networked workstation 542 may include a display 544, one or more input devices 546 (e.g., a keyboard, a mouse), and a processor 548. The networked workstation 542 may be located within the same facility as the operator workstation 502, or in a different facility, such as a different healthcare institution or clinic.

The networked workstation 542 may gain remote access to the data processing server 514 or data store server 516 via the communication system 540. Accordingly, multiple networked workstations 542 may have access to the data processing server 514 and the data store server 516. In this manner, magnetic resonance data, reconstructed images, or other data may be exchanged between the data processing server 514 or the data store server 516 and the networked workstations 542, such that the data or images may be remotely processed by a networked workstation 542.

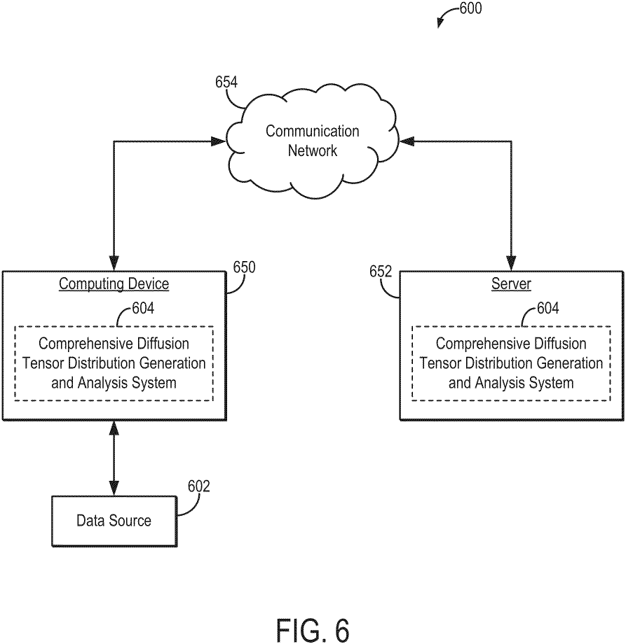

Referring now to FIG. 6, an example of comprehensive diffusion tensor distribution analysis system 600 is shown, which may be used in accordance with some aspects of the systems and methods described in the present disclosure. As shown in FIG. 6, a computing device 650 can receive one or more types of data (e.g., diffusion-weighted imaging data, CDTD data, diffusion tensor parameters for defining water pools, etc.) from data source 602. In some configurations, computing device 650 can execute at least a portion of a comprehensive diffusion tensor distribution generation and analysis system 604 to generate and/or analyze CDTD data based on data received from the data source 602. The diffusion imaging system 604 can implement the methods described herein, such as the method described in FIG. 2.

Additionally or alternatively, in some configurations, the computing device 650 can communicate information about data received from the data source 602 to a server 652 over a communication network 654, which can execute at least a portion of the comprehensive diffusion tensor distribution generation and analysis system 604. In such configurations, the server 652 can return information to the computing device 650 (and/or any other suitable computing device) indicative of an output of the comprehensive diffusion tensor distribution generation and analysis system 604.

In some configurations, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data.

In some configurations, data source 602 can be any suitable source of data (e.g., measurement data, images reconstructed from measurement data, processed image data), such as an MRI system, another computing device (e.g., a server storing measurement data, images reconstructed from measurement data, processed image data), and so on. In some configurations, data source 602 can be local to computing device 650. For example, data source 602 can be incorporated with computing device 650 (e.g., computing device 650 can be configured as part of a device for measuring, recording, estimating, acquiring, or otherwise collecting or storing data). As another example, data source 602 can be connected to computing device 650 by a cable, a direct wireless link, and so on. Additionally or alternatively, in some configurations, data source 602 can be located locally and/or remotely from computing device 650, and can communicate data to computing device 650 (and/or server 652) via a communication network (e.g., communication network 654).

In some configurations, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some configurations, communication network 654 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, and so on.

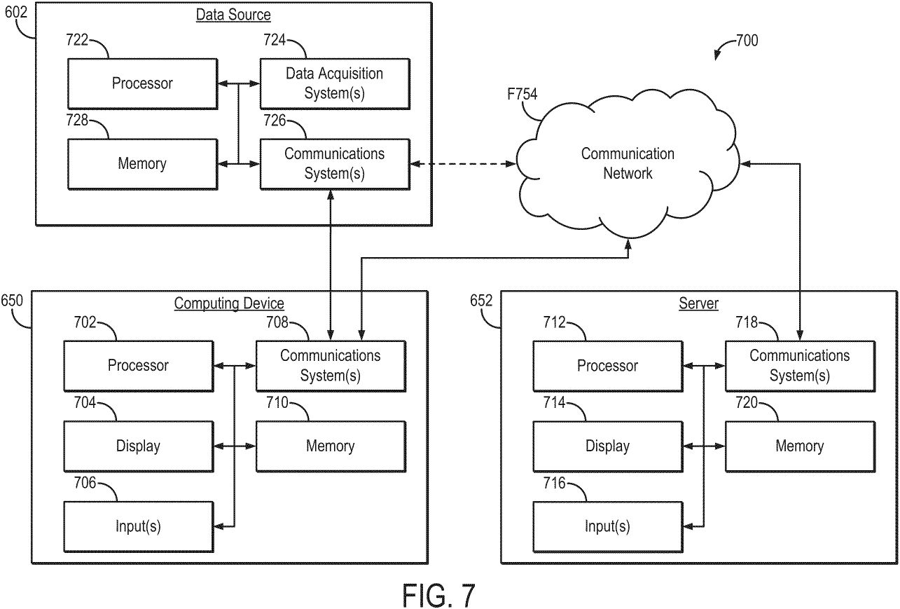

Referring now to FIG. 7, an example of hardware 700 that can be used to implement data source 602, computing device 650, and server 652 in accordance with some configurations of the systems and methods described in the present disclosure is shown.

As shown in FIG. 7, in some configurations, computing device 650 can include a processor 702, a display 704, one or more inputs 706, one or more communication systems 708, and/or memory 710. In some configurations, processor 702 can be any suitable hardware processor or combination of processors, such as a central processing unit (“CPU”), a graphics processing unit (“GPU”), and so on. In some configurations, display 704 can include any suitable display devices, such as a liquid crystal display (“LCD”) screen, a light-emitting diode (“LED”) display, an organic LED (“OLED”) display, an electrophoretic display (e.g., an “e-ink” display), a computer monitor, a touchscreen, a television, and so on. In some configurations, inputs 706 can include any suitable input devices and/or sensors that can be used to receive user input, such as a keyboard, a mouse, a touchscreen, a microphone, and so on.

In some configurations, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware, and/or software that can be used to establish a Wi-Fi connection, a Bluetooth connection, a cellular connection, an Ethernet connection, and so on.

In some configurations, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include random-access memory (“RAM”), read-only memory (“ROM”), electrically programmable ROM (“EPROM”), electrically erasable ROM (“EEPROM”), other forms of volatile memory, other forms of non-volatile memory, one or more forms of semi-volatile memory, one or more flash drives, one or more hard disks, one or more solid state drives, one or more optical drives, and so on. In some configurations, memory 710 can have encoded thereon, or otherwise stored therein, a computer program for controlling operation of computing device 650. In such configurations, processor 702 can execute at least a portion of the computer program to present content (e.g., images, user interfaces, graphics, tables), receive content from server 652, transmit information to server 652, and so on. For example, the processor 702 and the memory 710 can be configured to perform the methods described herein (e.g., the method of FIG. 1, the method of FIG. 2).

In some configurations, server 652 can include a processor 712, a display 714, one or more inputs 716, one or more communications systems 718, and/or memory 720. In some configurations, processor 712 can be any suitable hardware processor or combination of processors, such as a CPU, a GPU, and so on. In some configurations, display 714 can include any suitable display devices, such as an LCD screen, LED display, OLED display, electrophoretic display, a computer monitor, a touchscreen, a television, and so on. In some configurations, inputs 716 can include any suitable input devices and/or sensors that can be used to receive user input, such as a keyboard, a mouse, a touchscreen, a microphone, and so on.

In some configurations, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware, and/or software that can be used to establish a Wi-Fi connection, a Bluetooth connection, a cellular connection, an Ethernet connection, and so on.

In some configurations, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 720 can have encoded thereon a server program for controlling operation of server 652. In such configurations, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some configurations, the server 652 is configured to perform the methods described in the present disclosure. For example, the processor 712 and memory 720 can be configured to perform the methods described herein (e.g., the method of FIG. 1, the method of FIG. 2).

In some configurations, data source 602 can include a processor 722, one or more data acquisition systems 724, one or more communications systems 726, and/or memory 728. In some configurations, processor 722 can be any suitable hardware processor or combination of processors, such as a CPU, a GPU, and so on. In some configurations, the one or more data acquisition systems 724 are generally configured to acquire data, images, or both, and can include an MRI system. Additionally or alternatively, in some configurations, the one or more data acquisition systems 724 can include any suitable hardware, firmware, and/or software for coupling to and/or controlling operations of an MRI system. In some configurations, one or more portions of the data acquisition system(s) 724 can be removable and/or replaceable.

Note that, although not shown, data source 602 can include any suitable inputs and/or outputs. For example, data source 602 can include input devices and/or sensors that can be used to receive user input, such as a keyboard, a mouse, a touchscreen, a microphone, a trackpad, a trackball, and so on. As another example, data source 602 can include any suitable display devices, such as an LCD screen, an LED display, an OLED display, an electrophoretic display, a computer monitor, a touchscreen, a television, etc., one or more speakers, and so on.

In some configurations, communications systems 726 can include any suitable hardware, firmware, and/or software for communicating information to computing device 650 (and, in some configurations, over communication network 654 and/or any other suitable communication networks). For example, communications systems 726 can include one or more transceivers, one or more communication chips and/or chip sets, and so on. In a more particular example, communications systems 726 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some configurations, memory 728 can include any suitable storage device or devices that can be used to store instructions, values, data, or the like, that can be used, for example, by processor 722 to control the one or more data acquisition systems 724, and/or receive data from the one or more data acquisition systems 724; to generate images from data; present content (e.g., data, images, a user interface) using a display; communicate with one or more computing devices 650; and so on. Memory 728 can include any suitable volatile memory, non-volatile memory, storage, or any suitable combination thereof. For example, memory 728 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some configurations, memory 728 can have encoded thereon, or otherwise stored therein, a program for controlling operation of medical image data source 602. In such configurations, processor 722 can execute at least a portion of the program to generate images, transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone, etc.), and so on.

In some configurations, any suitable computer-readable media can be used for storing instructions for performing the functions and/or processes described herein. For example, in some configurations, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “controller,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

As used herein, the phrase “at least one of A, B, and C” means at least one of A, at least one of B, and/or at least one of C, or any one of A, B, or C or combination of A, B, or C. A, B, and C are elements of a list, and A, B, and C may be anything contained in the Specification.

The present disclosure has described one or more preferred embodiments, and it should be appreciated that many equivalents, alternatives, variations, and modifications, aside from those expressly stated, are possible and within the scope of the invention.

1. A method for imaging diffusion using magnetic resonance imaging (MRI), the method comprising:
(a) accessing, with a computer system, diffusion-weighted imaging data acquired from a region-of-interest in a subject using an MRI system;
(b) generating a comprehensive diffusion tensor distribution (CDTD) from the diffusion-weighted imaging data using the computer system, wherein the CDTD indicates a proportion of water molecules in each voxel in the region-of-interest whose diffusion corresponds to each of a plurality of diffusion tensors; and
(c) outputting the CDTD with the computer system.

2. The method of claim 1, wherein generating the CDTD comprises defining a maximum diffusion tensor value, defining a minimum diffusion tensor value, and constraining the plurality of diffusion tensors by the maximum diffusion tensor value and the minimum diffusion tensor value.

3. The method of claim 1, wherein each of the plurality of diffusion tensors is described by eigenvalues, λ₁, λ₂, and λ₃, and wherein generating the CDTD comprises constraining the plurality of diffusion tensors by λ₁ ≤ λ₂ ≤ λ₃.

3. The method of claim 1, wherein generating the CDTD comprises selecting the plurality of diffusion tensors by uniformly discretizing a range of candidate diffusion tensors.

4. The method of claim 1, wherein generating the CDTD comprises selecting the plurality of diffusion tensors by logarithmically discretizing a range of candidate diffusion tensors.

5. The method of claim 1, wherein outputting the CDTD comprises generating a water pool image from the CDTD and outputting the water pool image with the computer system, wherein the water pool image depicts water diffusion associated with a subset of the plurality of diffusion tensors.

6. The method of claim 5, wherein the water pool image is generated by masking the CDTD to select the subset of the plurality of diffusion tensors.

7. The method of claim 6, wherein generating the water pool image comprises:
selecting a set of diffusion tensor parameters defining the subset of the plurality of diffusion tensors;
masking the CDTD to select the subset of the plurality of diffusion tensors having parameters that satisfy the set of diffusion tensor parameters; and
weighting voxels in the water pool map based on a number of diffusion tensors in the subset of the plurality of diffusion tensors for each voxel.

8. The method of claim 7, wherein selecting the set of diffusion parameters comprises defining a cutoff threshold D_{cut}, and wherein the set of diffusion tensor parameters comprises λ₁, λ₂, λ₃ ≥ D_{cut} mm²/s and 3λ₁ ≥ λ₂, 3λ₂ ≥ λ₃.

9. The method of claim 7, wherein selecting the set of diffusion parameters comprises defining a cutoff threshold D_{cut}, and wherein the set of diffusion tensor parameters comprises λ₁ < D_{cut}, λ₂ < D_{cut}, and
$$\left({λ}_{1} + {λ}_{2}\right) < \frac{{λ}_{3}}{8} .$$
.

10. The method of claim 7, wherein selecting the set of diffusion parameters comprises defining a cutoff threshold D_{cut}, and wherein the set of diffusion tensor parameters comprises
$${λ}_{1} < {D}_{c u t} , {λ}_{2} > \frac{{λ}_{3}}{3} \text{, and} {λ}_{2} < 3 {λ}_{3} .$$
.

11. The method of claim 1, wherein outputting the CDTD comprises generating a classification image from the CDTD and outputting the classification image with the computer system, wherein the classification image depicts classified regions in the region-of-interest that are classified based on subsets of the plurality of diffusion tensors.

12. The method of claim 11, wherein generating the classification image comprises inputting the CDTD to a clustering algorithm, generating the classified image as an output, wherein each of the classified regions in the region-of-interest correspond to a different cluster of voxels generated by the clustering algorithm.

13. The method of claim 12, wherein the clustering algorithm comprises one of a hierarchical clustering algorithm, a k-means clustering algorithm, a bi-clustering algorithm, or a machine learning model-based clustering algorithm.

14. The method of claim 13, wherein the clustering algorithm is the machine learning model-based clustering algorithm and the machine learning model-based clustering algorithm comprises a neural network that has been trained on training data to receive CDTD data as an input and generate clusters of similar voxels as an output.

15. The method of claim 1, wherein outputting the CDTD comprises:
analyzing the CDTD, using the computer system, to assess different types of diffusion in tissues in the region-of-interest; and
generating, with the computer system, a report based on analyzing the CDTD, wherein the report indicates microstructure of the tissues in the region-of-interest.

16. The method of claim 15, wherein the report indicates a heterogeneity of the tissues in the region-of-interest.

17. The method of claim 15, wherein analyzing the CDTD comprises generating a plurality of water pool images from the CDTD and outputting the plurality of water pool images as part of the report, wherein each of the plurality of water pool images depicts water diffusion associated with a different subset of the plurality of diffusion tensors.

18. The method of claim 15, wherein analyzing the CDTD comprises generating a classification image from the CDTD and outputting the classification image as part of the report, wherein the classification image depicts classified regions in the region-of-interest that are classified based on subsets of the plurality of diffusion tensors.
